(19) United States
(12) Patent Application Publication
(10) Pub. No.: US 2023/0354936 A1
(43) Pub. Date: Nov. 9, 2023

(54) Fashion Compression Garment

(71) Applicant: Leslie-Michelle Lopez, Ramsey, NJ (US)

(72) Inventor: Leslie-Michelle Lopez, Ramsey, NJ (US)

(21) Appl. No.: 17/737,971

(22) Filed: May 5, 2022

(51) Int. Cl. A41D 31/18; A61F 5/30

(52) U.S. Cl. CPC A41D 31/18; A61F 5/30

(57) ABSTRACT

A fashion garment having one or more regions wherein at least one of the one or more regions is a compression region made of at least one elastic fiber material and at least one hard fiber material, each of the compression regions configured to fit snuggly around a body part of a wearer, the compression regions providing medium to high compression. The fashion garment constructed to be worn as everyday fashion apparel, eliminating the need for concealing separate undergarment body shapers.

### BACKGROUND

### Field of the Invention

The invention relates generally to fashion garments, and more specifically to fashion garments that are made at least in part of medium and high medical grade compression materials and are constructed and designed to be worn without the need for undergarments.

### Related Art

Compression garments are garments that fit tightly around the skin of a wearer, functioning to promote blood flow and increase the amount of oxygen to the muscles. Compression garments are generally known for assisting in faster recovery from athletic activities, for use by women suffering through pregnancy induced edema, for assisting with varicose veins, for post-surgery recovery, or by those looking to reduce the physiological impact of long-haul flights where inactive muscles can fatigue due to decreased blood flow to the extremities. Compression garments are designed to address the symptoms of a medical condition. Currently, most compression garments are not designed to be worn as fashion apparel but are designed solely for their functional purpose.

Compression garments are generally made of materials comprising of elastic fibers. The materials' ability to extend and elastically recover are the garments' most important characteristic, allowing the garments to exert continuous pressure on a wearer's body.

The purpose of shapewear garments is different from the purpose of compression garments. The function of shapewear garments is to smooth and enhance wearers' figures but shapewear garments don't generally provide the increased blood flood and oxygen to the muscles that compression garments provide. Powernet is one of the highest quality and most commonly used shapewear textiles and is typically lined with cotton to provide additional comfort to a wearer.

The invention is directed to fashion garments providing aesthetic enhancements as well as compression functionality in a single garment that can be worn as everyday fashion apparel eliminating the need for wearing undergarments. The fashion garments may be worn as everyday fashion apparel providing the functional technology of eliminating the necessity of concealing undergarment body shapers, heat and irritation of added layers, providing the highest level of body support resulting in a more defined and contoured waistline and silhouette, accentuating curves, reducing inches, providing posture support, boosting blood support/circulation in the lower extremities, and post-surgical benefits including reduction in discomfort and swelling, all in one, single constructed garment.

### SUMMARY OF THE INVENTION

It is to be understood that the phraseology and terminology employed herein are for the purpose of the description and should not be regarded as limiting.

The invention is directed to fashion garments comprising one or more regions wherein at least one of the one or more regions is a compression region. Each compression region made of at least one elastic fiber material and at least one ‘hard’ fiber material and configured to provide medium to high compression when the fashion garment is worn.

These and other features of the invention will become readily apparent upon further review of the specification and drawings.

### BRIEF DESCRIPTION OF THE DRAWINGS

Embodiments will be described by way of example only, and not limitation, with reference to the accompanying drawings. The drawings are not necessarily drawn to scale and wherever possible, the same or like reference numbers are used throughout the drawings to refer to the same or like parts.

FIG. 1 is a front perspective view of a fashion garment according to embodiments of the present invention;

FIG. 2 is a front perspective view of a fashion garment according to embodiments of the present invention; and

FIG. 3 is a front perspective view of a fashion garment according to embodiments of the present invention.

### DETAILED DESCRIPTION

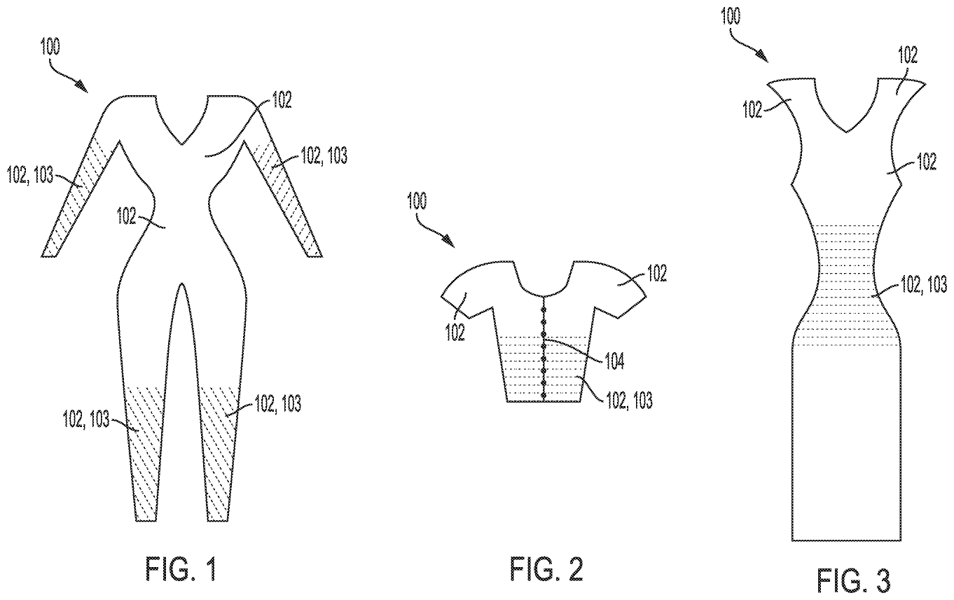

Referring now to the figures where similar reference characters denote similar elements throughout the figures, FIGS. 1 through 3 show a fashion garment 100 having one or more regions 102 wherein at least one of the one or more regions is a compression region 103. The compression region 103 made of at least one elastic fiber material and at least one ‘hard’ fiber material and configured to provide a wearer with medium to high compression.

The fashion garment 100 may include one or more closures 104 for securing the fashion garment 100 around a wearer's body. The one or more closures may be hook and look, hook and eye, zip fastener, magnetic fastener, or adhesive fastener, although any type of fastener means known in the art may be used. The location of the closures 104 may vary. The closures 104 may be on the front, the side, the back, or in any other suitable location of the fashion garment 100. The fashion garment 100 may be free of closures 104 and instead configured so that a wearer may put the garment on without the need to open the garment by way of a closure 104.

The fashion garment 100 may be constructed in whole or in part from a four-way stretch fabric blend having moderate to firm compression capabilities and enhanced durability, with the ability to recover after long periods of wear and lined with cotton or any other similar material for movement, comfort and breathability. The compression regions 103 fit tightly around a corresponding region of a wearer's body and provide a wearer with moderate to firm compression with pressures ranging from 8 mm/Hg to 30 mm/Hg. Some commercially available fabrics ideal for use for the compression regions may include PowerNet and ComfortWeave®. The compression regions 103 include a blend of ‘hard’ and ‘soft’ fibers. Soft fibers are highly elastic like elastane while hard non-elastic fibers may include nylon and similar fibers. By way of example, PowerNet is comprised of 25% Spandex, 75% Nylon and ComfortWeave® is comprised of 49% Spandex, 51% Nylon.

The fashion garment 100 may be constructed in whole or in part from materials that provide moisture wicking to draw sweat away from the skin. Such materials may include polyester, polypropylene, merino wool, wool, nylon, micromodal, bamboo, or other moisture wicking materials. The fashion garment 100 may include an antibacterial and odor resistant application to minimize odor and bacterial growth, such as active silver, or zinc and copper containing chitosan fibers. The fashion garment 100 may include materials that provide an Ultraviolet Protection Factor (UPF) of 30 or more for protection against the sun's harmful rays. The UPF measures the amount of ultraviolet (UV) radiation that can penetrate fabric and reach a wearer's skin. Dark and bright colors, and densely woven fabrics, make it more difficult for the sun's rays to reach a wearer's skin. Fabrics treated with chemical UV absorbers or dyes prevent some penetration from UV rays.

The fashion garment 100 may be in the form of a top, a bottom, a dress, a bodysuit, swimwear, or other item of clothing. The fashion garment 100 is preferably seamless throughout with no closures 104 but may comprise of closures 104. The seamless construction is beneficial because seams can be irritating to the wearer's skin and may increase sweat. Because of its moisture wicking and seamless features, the fashion garment 100 replaces the need for undergarments. The fashion garment 100 may provide different levels of compression in different regions. For example, as shown in FIG. 1, embodiments of the fashion garment 100 may comprise of two arm regions 102 and two leg regions 102 all of which are compression regions 103. The arm compression regions 103 may have a medium compression whereas the leg compression regions 103 may have a high compression allowing a wearer to receive varying levels of compression in a single garment 100 to target certain issues that are specific to specific area of the body. The fashion garment 100 may be constructed in such a way that a wearer receives shapewear benefits from the fashion garment 100 as a whole and also receives compression benefits from the compression regions 103 in certain regions of their body and in varying intensities ranging from moderate to high. In exemplary embodiments, the fashion garment 100 is a seamless garment providing shapewear benefits throughout and compression benefits with high compression in at least the midsection and leg regions, where the garment comprises of these regions. However, exemplary embodiments may comprise of high compression in the midsection only where no leg regions exist, or vice versa. Similarly, as shown by way of example in FIG. 2, the fashion garment 100 may be in the form of a top having a single compression region 103 about the midsection area and closures 104 for securing the fashion garment around a wearer's body. As shown by way of example in FIG. 3, the fashion garment 100 may be in the form of a seamless dress, free of closures, and having a single compression region about the midsection of the garment 100.

The invention may be constructed by sewing, gluing, or other suitable means for constructing seamless garments. The invention may include attached pieces of fabric acting as ornamentation. The attached pieces of fabric may vary in type, for example, cotton, nylon, silk, wool. The attached pieces of fabric may also vary in style, (neckline, sleeve length, color), and in ornamentation. The attached pieces of fabric may serve a functional purpose aside from the combined compression and shapewear purpose disclosed by the invention, or may serve no functional purpose.

The fashion garment 100 is constructed to be worn as everyday fashion apparel, eliminating the need for concealing separate undergarment body shapers, eliminating the heat and irritation of added layers, providing the highest level of body support resulting in a more defined and contoured waistline and silhouette, accentuating curves, reducing inches, providing posture support, boosting blood support/circulation in the lower extremities, and post-surgical benefits including reduction in discomfort and swelling, all in one single constructed garment. The fashion garment 100 is engineered to improve circulation and hold muscles in the correct anatomical position; all of which are integral to minimizing muscle damage from both physical exercise and the general wear and tear of day-to-day activity.

A preferred method of manufacturing the fashion garment 100 is by way of a ball-point machine needle and a three (3) step zig-zag stitch on a standard sewing machine. An overlock machine on side seams may also be used, where seams exist. Polyester thread is also highly recommended when sewing with any type of elastomer fabric. Polyester thread is stronger than cotton. Cotton is likely to break as it cannot withstand the stretch experienced by shapewear and compression garments when in use. The ability of the fabric to stretch is a vital feature in all aspects of manufacturing the present invention including the type of thread used to construct the fashion garment 100.

What is claimed is:

1. A fashion garment comprising:
one or more regions wherein at least one of the one or more regions is a compression region made of at least one elastic fiber material and at least one hard fiber material, each of the compression regions configured to fit tightly around and apply pressure to a region of a wearer's body, the compression regions providing medium to high compression.

2. The fashion garment of claim 1, wherein the entire garment is seamless.

3. The fashion garment of claim 1, further comprising one or more closures configured for securing the fashion garment around a wearer's body.

4. The fashion garment of claim 1, wherein the one or more regions that are not compression regions are configured to enhance a wearer's body shape.

5. A fashion garment comprising:
one or more regions wherein at least two of the one or more regions are compression regions made of at least one elastic fiber material and at least one hard fiber material, the compression regions each configured to fit tight around and apply pressure to a region of a wearer's body, wherein a first of the at least two compression regions provides medium compression and a second of the at least two compression regions provides high compression.

6. The fashion garment of claim 5, wherein the entire garment is seamless.

7. The fashion garment of claim 5, further comprising one or more closures configured for securing the fashion garment around a wearer's body.

8. The fashion garment of claim 5, wherein the one or more regions that are not compression regions are configured to enhance a wearer's body shape.
